(19) United States
(12) Patent Application Publication
(10) Pub. No.: US 2018/0199833 A1
(43) Pub. Date: Jul. 19, 2018

(54) DEVICE FOR CALCULATING SYSTOLIC BLOOD PRESSURE USING PULSE TRANSIT TIME AND METHOD THEREFOR

(71) Applicant: UNIVERSITY OF ULSAN FOUNDATION FOR INDUSTRY COOPERATION, Ulsan (KR)

(72) Inventors: Sung-Hoon KIM, Seoul (KR); Gyu-Sam HWANG, Seoul (KR)

(73) Assignee: UNIVERSITY OF ULSAN FOUNDATION FOR INDUSTRY COOPERATION, Ulsan (KR)

(21) Appl. No.: 15/745,125

(22) PCT Filed: Jul. 14, 2016
(86) PCT No.: PCT/KR2016/007699

§ 371 Date: Jan. 15, 2018

(30) Foreign Application Priority Data
Jul. 14, 2015 (KR) 10-2015-0099804

(51) Int. Cl. A61B 5/021; A61B 5/0402

(52) U.S. Cl. CPC A61B 5/02125; A61B 5/1455; A61B 5/04023

(57) ABSTRACT

A method for calculating a systolic blood pressure includes receiving data regarding pulse transit time (PTT) and a systolic blood pressure (SBP) of a patient group, extracting a correlation between parameters (a, y₀) by analyzing results obtained by applying the data regarding the pulse transit time (PTT) and the systolic blood pressure (SBP) of the patient group to a PTT-SBP (Pulse Transit Time-Systolic Blood Pressure) relationship (SBP=a*PTT⁻¹+y₀), receiving data regarding a first pulse transit time (PTT) and a systolic blood pressure (SBP) of a measurement subject, and acquiring unique parameter values (a, y₀) of the measurement subject by applying the correlation between the parameters (a, y₀) and the data regarding the first pulse transit time (PTT) and the systolic blood pressure (SBP) of the measurement subject to the PTT-SBP relationship.

### ACKNOWLEDGEMENTS

Researches related to this Application were supported by the Korea Ministry of Science and ICT under the following projects.

Project number: 2016M3A9E8941259

Name of department: Ministry of Science and ICT

Research management institution: National Research Foundation of Korea

Research business name: Development of biomedical technology

Research project name: In-hospital smart monitoring system for vulnerable patients out of surveillance area

Main Institution: Asan Medical Center

Research period: Aug. 1, 2017 to May 31, 2018

Contribution rate: 50%

Project number: 2016R1C1B1012164

Name of department: Ministry of Science and ICT

Research management institution: National Research Foundation of Korea

Research business name: Personal Basic Research

Research project name: Development of non-invasive cardiovascular and respiratory monitoring system for critical patients receiving general anesthesia

Main Institution: University of Ulsan

Research period: Jun. 1, 2017 to Mar. 31, 2018

Contribution rate: 50%

### BACKGROUND

### (a) Field of the Invention

The present invention relates to a device for calculating a systolic blood pressure using pulse transit time and a method therefor, and particularly to a device for calculating a systolic blood pressure which calculates a unique characteristic parameter of a patient by receiving a pulse transit time of a measurement subject once without body measurement information of the patient and calculates an absolute value and a trend of change of the systolic blood pressure on the basis of the characteristic parameter.

### (b) Description of the Related Art

It is well known that pulse transit time (PTT) measured by combining a pulse oximeter and an electrocardiogram signal is inversely proportional to a systolic blood pressure (SBP).

In general, a relationship between the pulse transit time (PTT) and the systolic blood pressure (SBP) can be represented by SBP=a*PTT⁻¹+y₀, and since a and y₀ which are parameter values inherently differ and greatly deviate for each person, it is known to be difficult to predict.

Since there are two mathematically unknown numbers a and y₀, the unique values of a and y₀ of the patient can be obtained by measuring the actual systolic blood pressure (SBP) and pulse transit time (PTT) twice. However, if the systolic blood pressure (SBP) and the pulse transit time (PTT) do not change significantly even after several consecutive measurements, each measured value is substantially constant within an error range, and thus, it is impossible to know the unique parameter values a and y₀ of a patient. Therefore, it is known that a change of blood pressure can be estimated to some extent by observing a trend of the pulse transit time, an absolute value of the blood pressure cannot be known by a general method, and the blood pressure can be approximated to the absolute value to some extent when calibrated with various subject-related parameters such as body measurement information, disease information, and demographic characteristics of a subject, but, it is not clear which relevant parameters have to be selected and how much weight the parameters have to be calibrated.

A technology of the background of the present invention is disclosed in Japanese Patent Application No. 10-0638696 (issued on Oct. 26, 2006).

### SUMMARY

An object of the present invention is to provide a device for calculating a systolic blood pressure which receives pulse transit time of a subject once, calculates an absolute value of a systolic blood pressure, and calculates the absolute value and a trend of change of the systolic blood pressure, in a state of complete absence of prior information that can be calibrated, such as body measurement information, disease information, and demographic characteristics of the subject, and a method therefor.

According to one embodiment of the present invention, a method for calculating a systolic blood pressure includes receiving data regarding pulse transit time (PTT) and a systolic blood pressure (SBP) of a patient group, extracting a correlation between parameters (a, y₀) regarding a PTT-SBP (Pulse Transit Time-Systolic Blood Pressure) relationship (SBP=a*PTT⁻¹+y₀) by analyzing the data regarding the pulse transit time (PTT) and the systolic blood pressure (SBP), receiving data regarding a first pulse transit time (PTT) and a systolic blood pressure (SBP) of a measurement subject, and acquiring unique parameter values (a, y₀) of the measurement subject by applying the correlation between the parameters (a, y₀) and the data regarding the first pulse transit time (PTT) and the systolic blood pressure (SBP) of the measurement subject to the PTT-SBP relationship.

The receiving of the data of the patient group may be receiving data regarding pulse transit time (PTT) and a systolic blood pressure (SBP) of a patient undergoing induction of general anesthesia.

The extracting of the correlation between the parameters (a, y₀) may include extracting unique parameter values (a, y₀) of each patient regarding the PTT-SBP relationship by performing regression analysis of the data regarding the pulse transit time (PTT) and the systolic blood pressure (SBP) of the patient group; and extracting the correlation between the parameters (a, y₀) of the PTT-SBP relationship by performing the regression analysis of the extracted unique parameter values (a, y₀) of each patient.

In the correlation between the parameters (a, y₀), the correlation may be represented by an equation (y₀=118.87−3.25a).

The method for calculating a systolic blood pressure may further includes receiving a second pulse transit time (PTT) of the measurement subject, and calculating at least one of an absolute value and a trend of change of the systolic blood pressure (SBP) of the measurement subject by applying the second pulse transit time (PTT) and the unique parameter values (a, y₀) of the measurement subject to the PTT-SBP relationship.

According to another embodiment of the present invention, a device for calculating a systolic blood pressure includes a data input unit that receives data regarding pulse transit time (PTT) and a systolic blood pressure (SBP) of a patient group and data regarding pulse transit time (PTT) and a systolic blood pressure (SBP) of a measurement subject, a correlation extraction unit that extracts a correlation between parameters (a, y₀) regarding a PTT-SBP (Pulse Transit Time-Systolic Blood Pressure) relationship (SBP=a*PTT⁻¹+y₀) by analyzing the data regarding the pulse transit time (PTT) and the systolic blood pressure (SBP) of the patient group, and a unique parameter acquisition unit that acquires unique parameter values (a, y₀) of the measurement subject by applying the correlation between the parameters (a, y₀) and the data regarding the first pulse transit time (PTT) and the systolic blood pressure (SBP) of the measurement subject to the PTT-SBP relationship.

As such, according to the present invention, only one measurement of a systolic blood pressure (SBP) and pulse transit time (PTT) of a patient makes it possible to acquire the patient's unique parameters (a, y₀) regarding a PTT-SBP relationship with a very high level of accuracy, in a state of complete absence of prior information regarding a measurement subject, such as body measurement information, disease information, and demographic characteristics. Therefore, subsequent measurement of the patient's pulse transit time (PTT) alone makes it possible to calculate not only a trend of change of systolic blood pressure (SBP), but also an absolute value of the systolic blood pressure (SBP).

### BRIEF DESCRIPTION OF THE DRAWINGS

FIG. 1 is a configuration diagram of a device for calculating a systolic blood pressure according to an embodiment of the present invention.

FIG. 2 is a flowchart illustrating a systolic blood pressure calculating method according to an embodiment of the present invention.

FIG. 3 is a graph regarding pulse transit time and a systolic blood pressure of a patient undergoing induction of anesthesia according to the embodiment of the present invention.

FIG. 4 is a graph illustrating pulse transit times, systolic blood pressures, and a regression line of one patient according to the embodiment of the present invention.

FIG. 5 is a graph illustrating unique parameter values and a regression line for each patient according to the embodiment of the present invention.

FIG. 6 illustrates graphs in which a systolic blood pressure calculated according to the embodiment of the present invention is compared with a systolic blood pressure measured through insertion into an arterial tube.

FIG. 7 is a Bland-Altman Plot of the systolic blood pressure calculated according to the embodiment of the present invention and the systolic blood pressure measured through the insertion into the arterial tube.

### DETAILED DESCRIPTION

Hereinafter, embodiments of the present invention will be described in detail with reference to the accompanying drawings so as to be easily performed by those skilled in the art to which the present invention belongs. The present invention may, however, be embodied in many different forms and should not be limited to the embodiments set forth herein. In order to clearly describe the present invention, parts irrespective of the description are omitted, and like parts are denoted by like reference numerals or symbols throughout the specification.

Throughout the specification, when a portion is referred to as “including” a certain element, it means that the portion can further include another element, without excluding other elements unless described otherwise in particular.

Hereinafter, embodiments of the present invention will be described in detail with reference to the accompanying drawings so as to be easily performed by those skilled in the art in the present invention belongs.

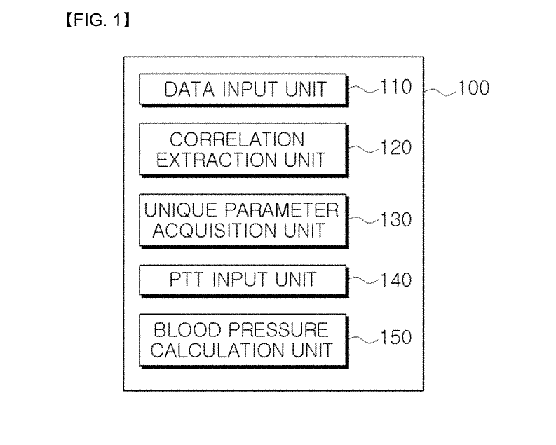

First, a device 100 for calculating a systolic blood pressure according to an embodiment of the present invention will be described with reference to FIG. 1. FIG. 1 is a configuration diagram of the device for calculating a systolic blood pressure according to the embodiment of the present invention.

The device 100 for calculating a systolic blood pressure according to the embodiment of the present invention includes a data input unit 110, a correlation extraction unit 120, and a unique parameter acquisition unit 130, and further includes a PTT input unit 140 and a blood pressure calculation unit 150.

Each configuration of the device 100 for calculating a systolic blood pressure will be specifically described. First, the data input unit 110 receives data regarding pulse transit time (PTT) and a systolic blood pressure (SBP) of a patient group Receive. In addition, the data input unit 110 receives data regarding a first pulse transit time (PTT) and the systolic blood pressure (SBP) of a measurement subject.

Here, the patient group includes a group of patients undergoing induction of general anesthesia. In addition, the data input unit 110 may receive measured data through an external measurement instrument and may receive the measured data through a measurement instrument included in the device 100 for a systolic blood pressure.

Next, the correlation extraction unit 120 extracts a correlation between parameters (a, y₀) of a PTT-SBP (Pulse Transit Time-Systolic Blood Pressure) relationship. At this time, the PTT-SBP relationship is as follows.

SBP=a×PTT⁻¹+y₀ [Equation 1]

Here, SBP means a systolic blood pressure, PTT means pulse transit time, and a and y₀ mean parameters of the PTT-SBP relationship.

In order to extract the correlation between the parameters (a, y₀) of the PTT-SBP relationship, the correlation extraction unit 120 analyzes data regarding the pulse wave propagation time (PTT) and the systolic blood pressure (SBP) of the patient group received from the data input unit 110, and extracts parameter values of the PTT-SBP relationship corresponding to data of each patient. Thereafter, the correlation extraction unit 120 analyzes the parameter values of the PTT-SBP relational ship corresponding to the extracted data of each patient and extracts the correlation of the PTT-SBP relationship.

At this time, the correlation extraction unit 120 can use a statistical analysis method for analyzing the pulse transit time (PTT) and the systolic blood pressure (SBP) of the patient group and for analyzing the parameter values (a, y₀) of the PTT-SBP relationship corresponding to the data of each patient, and the statistical analysis method includes a regression analysis method.

In addition, the unique parameter acquisition unit 130 acquires the unique parameter values (a, y₀) of a measurement subject regarding the PTT-SBP relationship. Specifically, the unique parameter acquisition unit 130 acquires the unique parameter values (a, y₀) of the measurement subject by applying the correlation between the parameters of the PTT-SBP relationship extracted by the correlation extraction unit 120 and the data regarding the first pulse transit time (PTT) and the systolic blood pressure (SBP) of the measurement subject to the PTT-SBP relationship.

Meanwhile, the pulse transit time (PTT) can be calculated by using an electrocardiogram signal measured from an electrocardiogram measurement sensor and a pulse signal measured from a pulse measurement sensor. Specifically, the pulse transit time (PTT) can be calculated through a difference between time of a peak point of an electrocardiogram signal generated by the same heartbeat and time of a maximum upslope point obtained by differentiating a pulse.

Next, the PTT input unit 140 receives a second pulse transit time (PTT) of a measurement subject. At this time, the PTT input unit 140 can receive the second pulse transit time (PTT) of the measurement subject measured through an external measurement instrument and can receive the second pulse transit time (PTT) of the measurement subject measured through a measurement instrument included in device 100 for calculating a systolic blood pressure.

Next, the blood pressure calculation unit 150 calculates an absolute value of the systolic blood pressure SBP corresponding to the second pulse transit time (PTT) of a measurement subject transmitted from the PTT input unit 140. In addition, the blood pressure calculation unit 150 can calculate a trend of change of the systolic blood pressure (SBP). In order to calculate at least one of the absolute value and the trend of change of the systolic blood pressure (SBP), the blood pressure calculation unit 150 applies the pulse transit time (PTT) of the measurement subject transmitted from the PTT input unit 140 and the unique parameter values (a, y₀) of the measurement subject transmitted from the unique parameter acquisition unit 130 to the PTT-SBP relationship.

According to the embodiment of the present invention, the device 100 for calculating a systolic blood pressure can be realized by including the data input unit 110, the unique parameter acquisition unit 130, the PTT input unit 140, and the blood pressure calculation unit 150, and the correlation between the parameters (a, y₀) of the PTT-SBP relationship extracted by the correlation extraction unit 120 can be stored in the device 100 for calculating a systolic blood pressure according to the embodiment of the present invention.

In addition, according to another embodiment of the present invention, the device 100 for calculating a systolic blood pressure can be realized by including the PTT input unit 140 and the blood pressure calculation unit 150, and the unique parameter values (a, y₀) of the measurement subject acquired by applying the correlation between the parameters (a, y₀) of the PTT-SBP relationship extracted by the correlation extraction unit 120 and the data regarding the first pulse transit time (PTT) and the systolic blood pressure (SBP) of the measurement subject that is, the PTT-SBP relationship can be stored in the device 100 for calculating a systolic blood pressure according to the embodiment of the present invention.

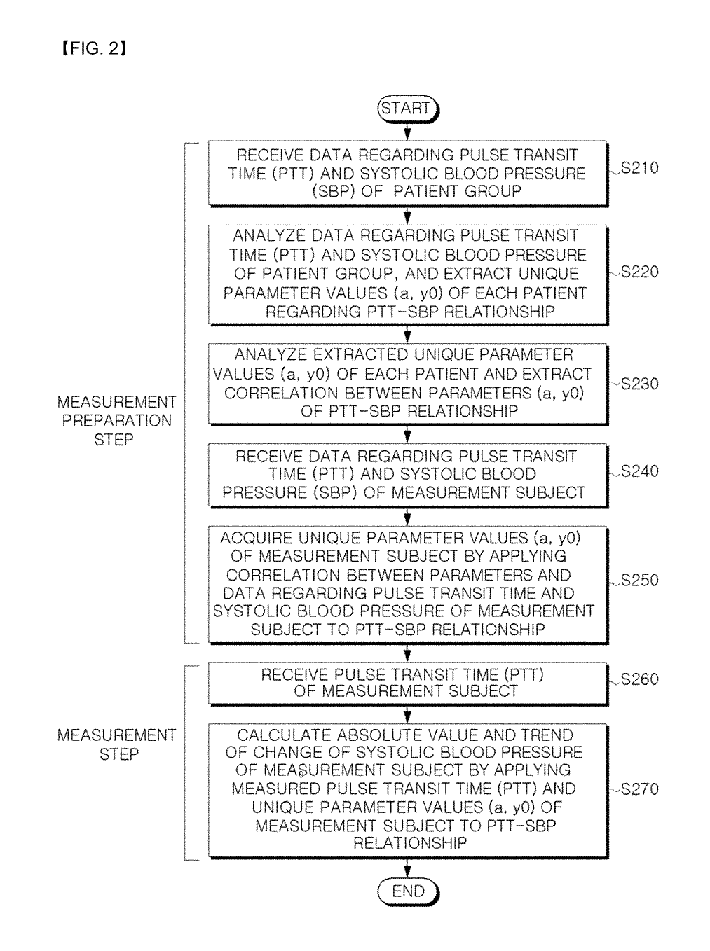

Hereinafter, a systolic blood pressure calculating method according to an embodiment of the present invention will be described with reference to FIGS. 2 to 5. FIG. 2 is a flowchart illustrating the systolic blood pressure calculating method according to the embodiment of the present invention.

The systolic blood pressure calculating method according to the embodiment of the present invention can be divided into blood pressure calculation preparation steps (S210 to S250) and blood pressure calculation steps (S260, S270). First, the blood pressure calculation preparation step will be specifically described. The blood pressure calculation preparation step is a process of extracting unique parameter values of a measurement subject regarding a PTT-SBP relationship and is to calculate an absolute value and a trend of change of a systolic blood pressure of the measure subject. That is, the device 100 for calculating a systolic blood pressure extracts the unique parameter values of the measurement subject regarding the PTT-SBP relationship through steps S210 to S250.

First, the device 100 for calculating a systolic blood pressure receives data regarding pulse transit time and a systolic blood pressure of a patient group (S210). At this time, the pulse transit time and the systolic blood pressure are measured every heartbeat of each patient, and correspond 1:1. Therefore, the data is calculated and input as a corresponding pair of measurement values from 70 to 90 number of times per minute, depending on a heart rate of a patient.

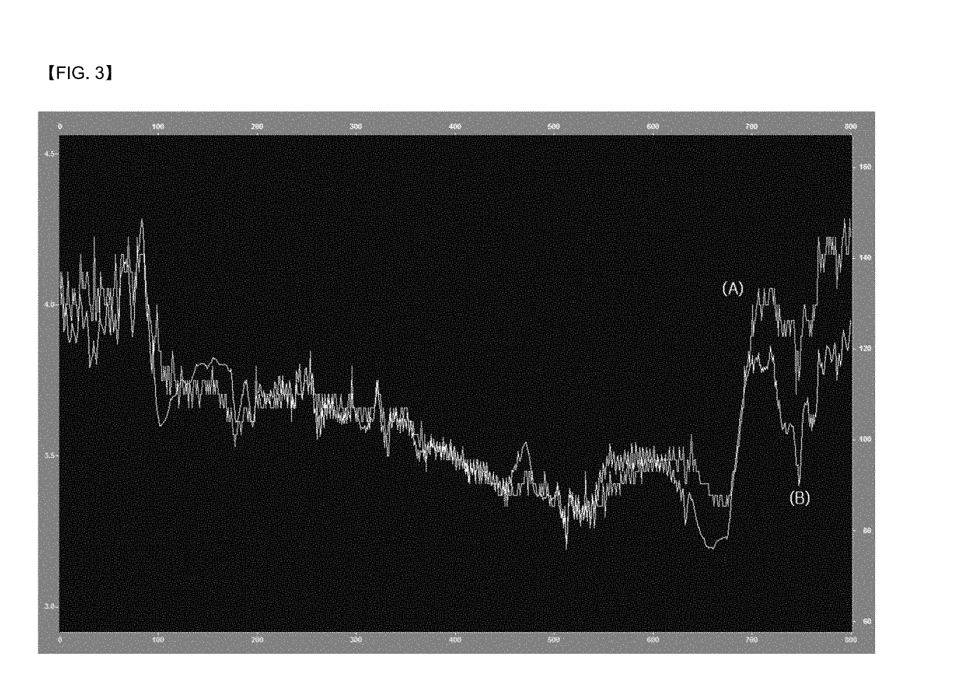

FIG. 3 is a graph regarding the pulse transit time and the systolic blood pressure of a patient undergoing induction of anesthesia according to the embodiment of the present invention. Among the waveforms illustrated in FIG. 3, (A) denotes the pulse transit time and (B) denotes the systolic blood pressure. As illustrated in FIG. 3, the pulse transit time and the systolic blood pressure have a similar manner in a trend of change with time. That is, as the systolic blood pressure decreases, the pulse transit time decreases, and as the systolic blood pressure increases, the pulse transit time increases. The trend of change is conspicuous because a change in blood pressure is made very rapidly in a short time in approximately 30% during induction of a general anesthesia. Particularly, the systolic blood pressure is an invasively measured value through insertion into an arterial tube, and thus, it can be said that the systolic blood pressure is the most accurate value (gold standard) at known technology levels.

Next, the device 100 for calculating a systolic blood pressure analyzes data regarding the pulse transit time and the systolic blood pressure of the patient group, and extracts unique parameter values of each patient regarding the PTT-SBP relationship (S220).

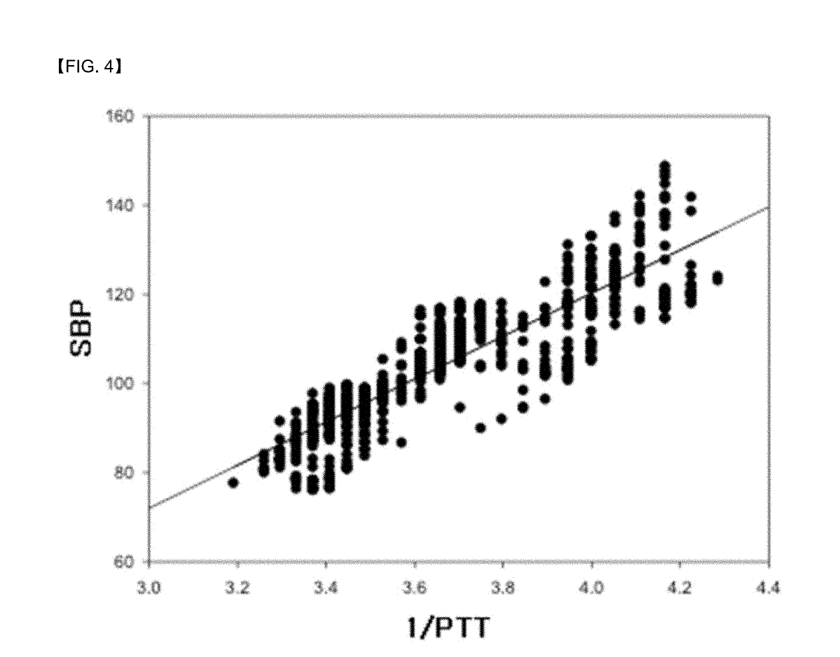

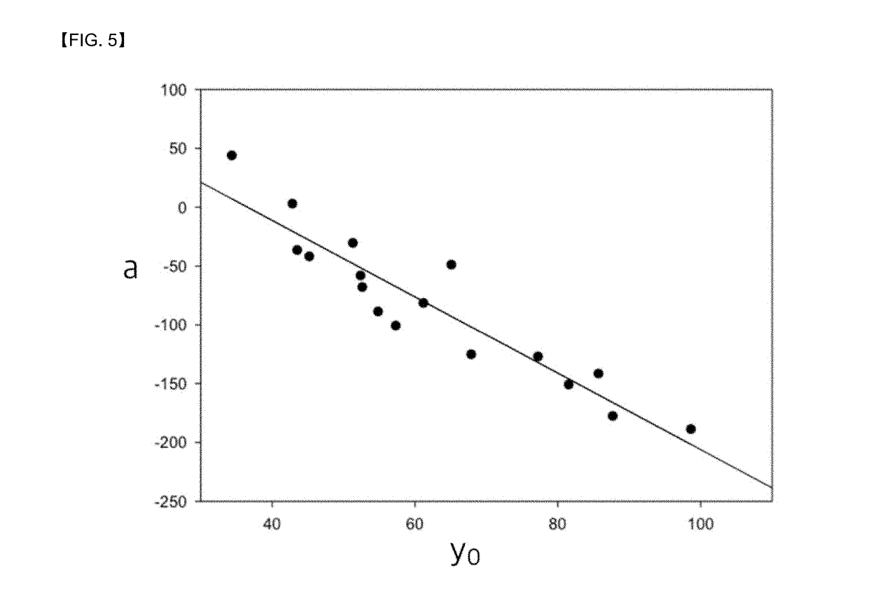

Hereinafter, steps of extracting the unique parameter values for each patient regarding the PTT-SBP relationship will be described in detail with reference to FIGS. 4 and 5. FIG. 4 is a graph illustrating the pulse transit times, the systolic blood pressures, and a regression line of one patient according to the embodiment of the present invention.

First, as illustrated in FIG. 4, the device 100 for calculating a systolic blood pressure plots the pulse transit time (PTT) and the systolic blood pressure (SBP) of one in a patient group on a two-dimensional coordinate plane having the pulse transit time (PTT) and the systolic blood pressure (SBP) as the X-axis and the SBP as the Y-axis. As can be seen from FIG. 4, the systolic blood pressure decreases and increases in a wide range at each pulse transit time.

Next, the device 100 for calculating a systolic blood pressure generates a line which is most suitable for points plotted on the two-dimensional coordinate plane having the PTT as the X-axis and the SBP as the Y-axis through a regression analysis, that is, a regression line. A line passing between the points illustrated in FIG. 4 is the regression line.

Then, the device 100 for calculating a systolic blood pressure analyzes the regression line and extracts unique parameter values of each patient regarding the PTT-SBP relationship.

Table 1 illustrates results of the regression analysis regarding FIG. 4.

TABLE 1

| R | Rsqr | Adj Rsqr | Standard Error of Estimate |
|---|---|---|---|
| 0.8774 | 0.7698 | 0.7695 | 6.7513 |

| | Coefficient | Std. Error | T | P | VIF |
|---|---|---|---|---|---|
| y₀ | −73.0075 | 3.4384 | −21.2327 | <0.000120 | 7.5090< |
| a | 48.3312 | 0.9357 | 51.6544 | <0.000120 | 7.5090< |

As illustrated in Table 1, the parameter (y₀) is −73.0075, the parameter (a) is 48.3312, and a correlation coefficient (R) is 0.8774 as results of the regression analysis of the patient, when assuming that the measured values are measured for 10 minutes, the measured data is calculated by the data regarding approximately 700 to 900 pulse wave transit times and systolic blood pressures which vary over a wide range. At this time, the correlation coefficient (R) is a numerical value indicating a degree of fitness of the regression line, and the closer the correlation coefficient is to 1, the higher the degree of fitness of the regression line is. It can be seen that the regression line illustrated in FIG. 4 has a high degree of fitness, in a case of the present patient.

Next, the device 100 for calculating a systolic blood pressure analyzes the extracted unique parameter values of each patient and extracts a correlation between the parameters of the PTT-SBP relationship (S230). At this time, the unique parameter values of each patient can be analyzed by using the regression analysis.

Hereinafter, steps of extracting the correlation between the parameters of the PTT-SBP relationship according to the embodiment of the present invention will be described with reference to FIG. 5. FIG. 5 is a graph illustrating the unique parameter values and the regression line of each patient according to the embodiment of the present invention.

First, the device 100 for calculating a systolic blood pressure plots the unique parameter values (a, y₀) of each patient determined in step S220 on a two-dimensional coordinate plane having a as the X-axis and a y₀ as the Y-axis. As illustrated in FIG. 5.

Next, the device 100 for calculating a systolic blood pressure generates a line which is most suitable for points plotted on the two-dimensional coordinate plane having a as the X-axis and y₀ as the Y-axis, that is, the regression line, among the unique parameter values measured in each patient through a regression analysis. A line passing between the dispersed points illustrated in FIG. 5 is the regression line.

Then, the device 100 for calculating a systolic blood pressure analyzes the regression line and extracts the correlation between the parameters of the PTT-SBP relationship.

Table 2 is a table illustrating resulted values generated by performing the regression analysis of the unique parameter values of a patient group using the device 100 for calculating a systolic blood pressure, according to the embodiment of the present invention.

TABLE 2

| R | Rsqr | Adj Rsqr | Standard Error of Estimate |
|---|---|---|---|
| 0.9359 | 0.8759 | 0.8676 | 23.0940 |

| | Coefficient | Std. Error | T | P | VIF |
|---|---|---|---|---|---|
| d | 118.8774 | 20.4870 | 5.8026 | <0.0001 | 13.3784< |
| c | −3.2505 | 0.3160 | −10.2879 | <0.0001 | 13.3784< |

As illustrated in Table 2, a slope (c) is −3.25, a slice (d) of a straight line is 118.87, and the correlation coefficient (R) is 0.9359 as results of the regression analysis of the unique parameter values. At this time, the correlation coefficient (R) is close to 1, and it can be seen that the regression line illustrated in FIG. 5 has a high degree of fitness. This is statistically significant results as results calculated from actually measured values of a total of 14,000 pairs or more, and thus, it is reasonable to apply the correlation to a general group.

When the correlation between the parameters is represented by using the above resulted values, the correlation between the parameters (a, y₀) represented by Equation (2) is extracted.

y₀=118.87−3.25a [Equation 2]

After the correlation between the parameters (a, y₀) is extracted in step S230, the device 100 for calculating a systolic blood pressure receives data regarding the first pulse transit time and the systolic blood pressure of the measurement subject (S240), and acquires the unique parameter values of a measurement subject regarding the PTT-SBP relationship by using the correlation between the parameters (a, y₀) and the data regarding the pulse transit time and systolic blood pressure of the measurement subject (S250).

A step of acquiring the unique parameter values of the measurement subject will be specifically described through the embodiment of the present invention. First, the device 100 for calculating a systolic blood pressure applies Equation (2) representing the correlation between the parameters (a, y₀) to the PTT-SBP relationship. Then, the device 100 for calculating a systolic blood pressure generates a result represented by Equation (3).

$$a = \frac{SBP - 118.87}{{PTT}^{- 1} - 3.25} \qquad Equation ( 3 )$$

Next, the device 100 for calculating a systolic blood pressure acquires the unique parameter value (a) of the measurement subject by applying the first pulse transit time and the systolic blood pressure of the measurement subject measure once to Equation (3).

In addition, the device 100 for calculating a systolic blood pressure inserts the acquired unique parameter value (a) of the measurement subject into Equation (2) representing the correlation between the parameters of the PTT-SBP relationship, and acquires the unique parameter value y₀ of the measurement subject.

For example, it is assumed that the first pulse transit time of the measurement subject is 0.270 [s] and the systolic blood pressure is 120 [mmHg]. Then, the device 100 for calculating a systolic blood pressure applies 0.270 [s] to the PTT and 120 to the SBP for Equation (2) and acquires 2.51 as the value a. In addition, the device 100 for calculating a systolic blood pressure applies the obtained value a which is (2.51) to Equation (1) and acquires 110.71 as the value y₀.

Hereinafter, a blood pressure calculation step according to the embodiment of the present invention will be specifically described. First, the blood pressure calculation step is a process of receiving the pulse transit time of the measurement subject after the blood pressure calculation preparation steps (S210 to S250), and of calculating at least one of an absolute value and a trend of change of the systolic blood pressure of the measurement subject corresponding thereto.

After acquiring the unique parameter values of the measurement subject through the step S250 of FIG. 2, the device 100 for calculating a systolic blood pressure receives the second pulse transit time of the measurement subject (S260). In addition, at least one of the absolute value and the trend of change of the systolic blood pressure is calculated by using the unique parameter value of the measurement subject acquired in step S250, the pulse transit time of the measurement subject measured in step S260, and the PTT-SBP relationship (S270).

For example, it is assumed that the pulse transit time measured in step S260 is 0.270 [s], and a is 2.51 and y₀ is 110.71 which are the unique parameter values of the measurement subject extracted in step S250. Then, the device 100 for calculating a systolic blood pressure derives the unique PTT-SBP relationship (SBP=1.50 PTT⁻¹+117.12) of the measurement subject by applying the unique parameter values to the PTT-SBP relationship, and thereafter, calculates a value of 120.00 [mmHg] as an absolute value of the systolic blood pressure by applying 0.270 [s] which is the pulse transit time of the measurement subject measured in step S250 to the unique PTT-SBP relationship (SBP=1.50 PTT⁻¹+117.12) of the measurement subject.

Next, a systolic blood pressure calculating method according to an embodiment of the present invention will be described.

First, the device 100 for calculating a systolic blood pressure receives data regarding the first pulse transit time (PTT) and the systolic blood pressure (SBP) of the measurement subject.

Then, the device 100 for calculating a systolic blood pressure acquires the unique parameter value (a) of the measurement subject by applying the input data regarding the first pulse transit time (PTT) and the systolic blood pressure (SBP) of the measurement subject to the PTT-SBP relationship (SBP=a*PTT⁻¹+y₀). At this time, since the correlation between the parameters is y₀=118.87−3.25a, the parameter value (a) is acquired by Equation (3)

$$( a = \frac{SBP - 118.87}{{PTT}^{- 1} - 3.25} ) .$$

In addition, the device 100 for calculating a systolic blood pressure receives the second pulse transit time (PTT) of the measurement subject.

Then, at least one of the absolute value and the trend of change of the systolic blood pressure (SBP) of the measurement subject is calculated by applying the second pulse transit time (PTT) and the unique parameter value a of the measurement subject to the PTT-SBP relationship.

In another embodiment of the present invention, the device 100 for calculating a systolic blood pressure receives the second pulse transit time (PTT) of the measurement subject.

In addition, at least one of the absolute value and the trend of change of the systolic blood pressure (SBP) of the measurement subject is calculated by applying the second pulse transit time (PTT) and the unique parameter value a of the measurement subject to the PTT-SBP relationship.

At this time, the unique parameter value (a) of the measurement subject can be stored in the device 100 for calculating a systolic blood pressure according to the present invention.

Meanwhile, the systolic blood pressure calculating method according to the embodiment of the present invention can be implemented in a form of a computer-readable recording medium in which a program for executing the systolic blood pressure calculating method is recorded.

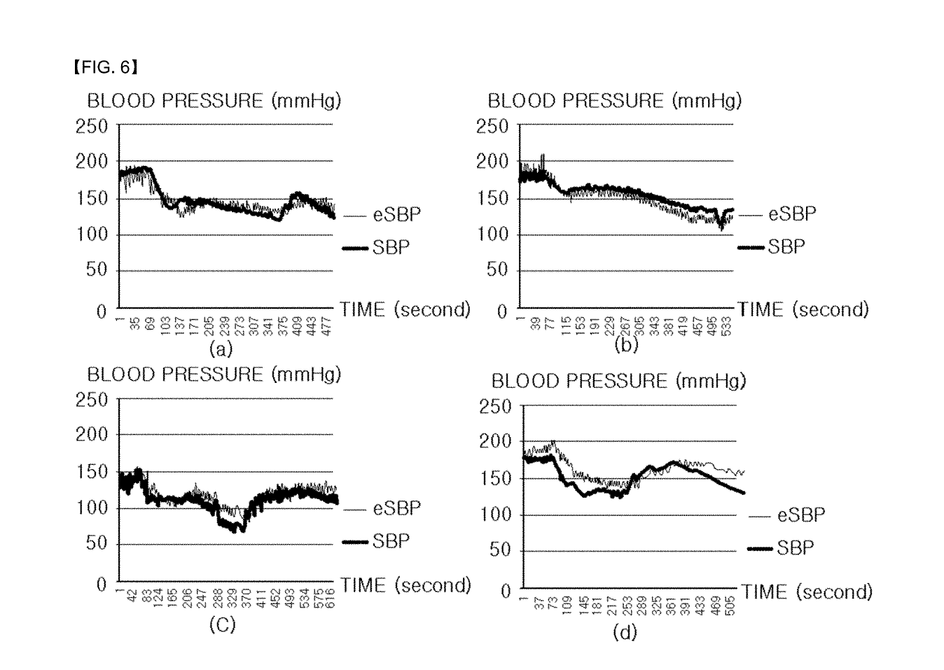

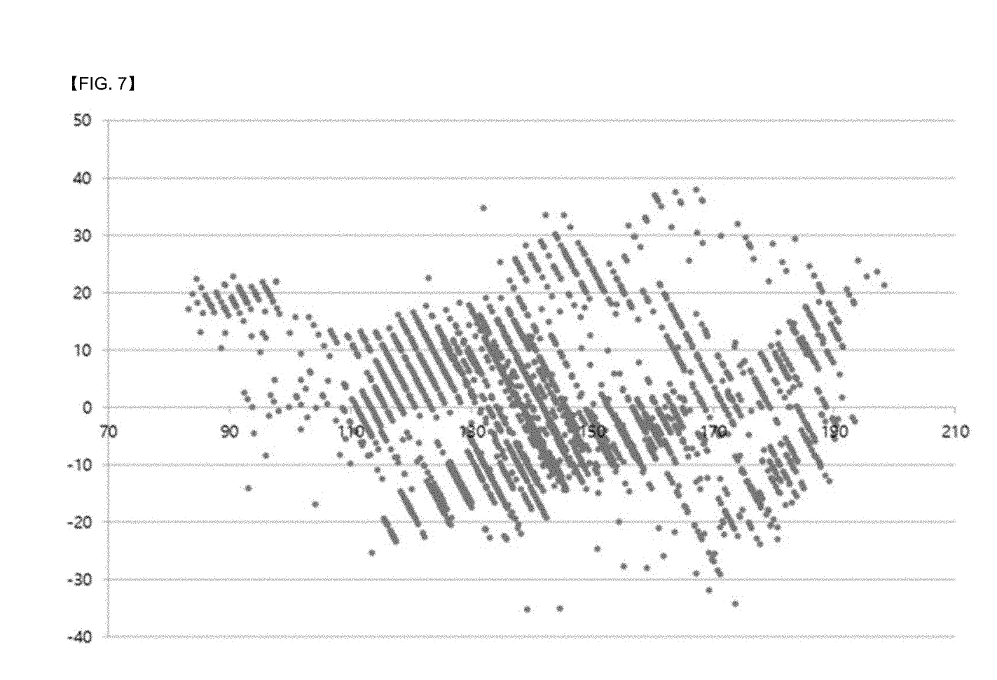

Hereinafter, accuracy of the systolic blood pressure calculated according to the embodiment of the present invention will be described with reference to FIGS. 6 and 7.

FIG. 6 illustrates graphs in which the systolic blood pressure calculated according to the embodiment of the present invention is compared with the systolic blood pressure measured through insertion of an arterial tube. In FIG. 6, eSBP represents the systolic blood pressure calculated according to the embodiment of the present invention, and SBP represents the systolic blood pressure measured through the insertion into the arterial tube. In general, an invasive blood pressure measurement value measured by inserting a catheter or the like into an arterial tube of a subject is more accurate than a value measured by using a non-invasive method, and thus, the comparison is made based on the invasive blood pressure measurement value in FIG. 6.

(a) to (d) of FIG. 6 are graphs of comparison between measurement subjects different from each other, and it can be seen that a pattern and a size of the systolic blood pressure calculated according to the embodiment of the present invention change in a similar manner to a pattern and a size of the invasive blood pressure measurement value, as illustrated (a) to (d) of FIG. 6.

FIG. 7 is a Bland-Altman Plot of the systolic blood pressure calculated according to the embodiment of the present invention and the systolic blood pressure measured through insertion into an arterial tube.

The Bland-Altman Plot is a graph illustrating a difference between values obtained by using two measurement methods. FIG. 7 graphically illustrates the difference between the systolic blood pressure and the invasive blood pressure measurement value calculated according to the embodiment of the present invention.

In FIG. 7, a horizontal axis represents an average value of the systolic blood pressure and the invasive blood pressure measurement value calculated according to the embodiment of the present invention, and a vertical axis represents a difference value between the systolic blood pressure and the invasive blood pressure measurement value calculated according to the embodiment of the present invention.

The Bland-Altman plot of FIG. 7 used data measured from a kidney transplant patient with hypertension, and 3336 data sets are used in which the systolic blood pressures and the invasive blood pressure measurement values calculated according to the embodiment of the present invention are paired.

Table 3 below illustrates results of the graph illustrated in FIG. 7.

TABLE 3

| | Number of Data Sets | |
|---|---|---|
| Error | within Error Range | Accuracy |
| Less than ±20 mmHg | 3077 | 92.3% |
| Less than ±15 mmHg | 2676 | 80.3% |
| Less than ±10 mmHg | 1959 | 58.8% |

As illustrated in Table 3, among the total of 3336 data sets, the number of data sets with an error of ±20 mmHg or less between the systolic blood pressure and the invasive blood pressure measurement value calculated according to the embodiment of the present invention is 3077 which accounts for 92.3% of the total. In addition, the number of data sets with an error of ±15 mmHg or less is 2676 which accounts for 80.3% of the total, and the number of data sets with an error of ±10 mmHg or less is 1959 which accounts for 58.8% of the total.

As described above, it can be seen that a numeric value of the systolic blood pressure calculated according to the embodiment of the present invention is accurately calculated within a small error range in comparison with the invasive blood pressure measurement value.

As described above, according to the embodiment of the present invention, only one measurement of a systolic blood pressure (SBP) and pulse transit time (PTT) of a patient makes it possible to acquire the patient's unique parameters (a, y₀) regarding a PTT-SBP relationship with a very high level of accuracy, in a state of complete absence of prior information regarding a measurement subject, such as body measurement information, disease information, and demographic characteristics. Therefore, subsequent measurement of the patient's pulse transit time (PTT) alone makes it possible to calculate not only a trend of change of systolic blood pressure (SBP), but also an absolute value of the systolic blood pressure (SBP). As such, it is not possible for a technology or related art to realize a technology of calculating a unique characteristic coefficient of the patient by one measurement without the body measurement information and specifying an absolute value of the systolic blood pressure based on the unique characteristic coefficient. A first reason is that a method of related art can calculate the unique characteristic coefficient of a patient, only when measurement information on a height, a body weight, an age, an arm circumference, and the like, and an accompanying disease state such as elasticity of a blood vessel or viscosity of blood are known, and a second reason is that it is difficult to apply the method of related art, because, even if the absolute value of the systolic blood pressure is calculated by one measurement, it is not known at all whether or not the characteristic value of the patient previously calculated according to a degree of subsequent change of the blood pressure accurately reflects a subsequent change in the blood pressure.

In addition, since the present invention calculates a trend of change and an absolute value of the systolic blood pressure in a non-invasive manner, not only a burden on a body of the patient is not imposed, but also a continuous measurement can be made for a long time.

In addition, since the present invention can be applied to a device, which is guaranteed to be mobile and portable, such as a wearable device, not only an in-hospital patient but also a person in outdoor activities have an advantage of measuring a blood pressure in real time.

While the present invention is described with reference to exemplary embodiments, the embodiments are merely examples, and it will be understood by those skilled in the art that various modifications and equivalent embodiments can be made. Accordingly, the true scope of the present invention should be determined by the technical idea of the appended claims.

1. A method for calculating a systolic blood pressure comprising:
receiving data regarding pulse transit time (PTT) and a systolic blood pressure (SBP) of a patient group;
extracting a correlation between parameters (a, y0) regarding a PTT-SBP (Pulse Transit Time-Systolic Blood Pressure) relationship (SBP=a PTT−1+y0) by analyzing the data regarding the pulse transit time (PTT) and the systolic blood pressure (SBP);
receiving data regarding a first pulse transit time (PTT) and a systolic blood pressure (SBP) of a measurement subject; and
acquiring unique parameter values (a, y0) of the measurement subject by applying the correlation between the parameters (a, y0) and the data regarding the first pulse transit time (PTT) and the systolic blood pressure (SBP) of the measurement subject to the PTT-SBP relationship.

2. The method for calculating a systolic blood pressure of claim 1,
wherein the receiving of the data of the patient group is receiving data regarding pulse transit time (PTT) and a systolic blood pressure (SBP) of a patient undergoing induction of general anesthesia.

3. The method for calculating a systolic blood pressure of claim 1,
wherein the extracting of the correlation between the parameters (a, y0) includes,
extracting unique parameter values (a, y0) of each patient regarding the PTT-SBP relationship by performing regression analysis of the data regarding the pulse transit time (PTT) and the systolic blood pressure (SBP) of the patient group; and
extracting the correlation between the parameters (a, y0) of the PTT-SBP relationship by performing the regression analysis of the extracted unique parameter values (a, y0) of each patient.

4. The method for calculating a systolic blood pressure of claim 1,
wherein, in the correlation between the parameters (a, y0), the correlation is represented by an equation (y0=118.87−3.25a).

5. The method for calculating a systolic blood pressure of claim 1, further comprising:
receiving a second pulse transit time (PTT) of the measurement subject; and
calculating at least one of an absolute value and a trend of change of the systolic blood pressure (SBP) of the measurement subject by applying the second pulse transit time (PTT) and the unique parameter values (a, y0) of the measurement subject to the PTT-SBP relationship.

6. A device for calculating a systolic blood pressure comprising:
a data input unit that receives data regarding pulse transit time (PTT) and a systolic blood pressure (SBP) of a patient group and data regarding pulse transit time (PTT) and a systolic blood pressure (SBP) of a measurement subject;
a correlation extraction unit that extracts a correlation between parameters (a, y0) regarding a PTT-SBP (Pulse Transit Time-Systolic Blood Pressure) relationship (SBP=a PTT−1+y0) by analyzing the data regarding the pulse transit time (PTT) and the systolic blood pressure (SBP) of the patient group; and
a unique parameter acquisition unit that acquires unique parameter values (a, y0) of the measurement subject by applying the correlation between the parameters (a, y0) and the data regarding the first pulse transit time (PTT) and the systolic blood pressure (SBP) of the measurement subject to the PTT-SBP relationship.

7. The device for calculating a systolic blood pressure of claim 6,
wherein the data input unit receives data regarding pulse transit time (PTT) and a systolic blood pressure (SBP) of a patient undergoing induction of general anesthesia.

8. The device for calculating a systolic blood pressure of claim 6,
wherein the correlation extraction unit extracts unique parameter values (a, y0) of each patient regarding the PTT-SBP relationship by performing regression analysis of the data regarding the pulse transit time (PTT) and the systolic blood pressure (SBP) of the patient group; and extracts the correlation between the parameters (a, y0) of the PTT-SBP relationship by performing the regression analysis of the extracted unique parameter values (a, y0) of each patient.

9. The device for calculating a systolic blood pressure of claim 6,
wherein in the correlation extraction unit, the correlation is represented by an equation (y0=118.87−3.25a).

10. The device for calculating a systolic blood pressure of claim 6, further comprising:
a PTT input unit that receives a second pulse transit time (PTT) of the measurement subject; and
a blood pressure calculation unit that calculates at least one of an absolute value and a trend of change of the systolic blood pressure (SBP) of the measurement subject by applying the second pulse transit time (PTT) and the unique parameter values (a, y0) of the measurement subject to the PTT-SBP relationship.

11. (canceled)

12. A device for calculating a systolic blood pressure comprising:
a data input unit that receives data regarding a first pulse transit time (PTT) and a systolic blood pressure (SBP) of a measurement subject;
a unique parameter acquisition unit that acquires a unique parameter value (a) of the measurement subject by applying the received data regarding the first pulse transit time (PTT) and the systolic blood pressure (SBP) of the measurement subject and a correlation between parameters (a, y0) regarding a PTT-SBP relationship (SBP=a PTT−1+y0) to the PTT-SBP relationship (SBP=a PTT−1+y0);
a PTT input unit that receives a second pulse transit time (PTT) of the measurement subject; and
a blood pressure calculation unit that calculates at least one of an absolute value and a trend of change of the systolic blood pressure (SBP) of the measurement subject by applying the second pulse wave transmission time (PTT) and the unique parameter value (a) of the measurement subject to the PTT-SBP relationship,
wherein the correlation between the parameters (a, y0) is extracted by analyzing data regarding pulse transit time (PTT) and a systolic blood pressure (SBP) of a patient group.

13. (canceled)

14. A device for calculating a systolic blood pressure comprising:
a PTT input unit that receives a second pulse transit time (PTT) of a measurement subject; and
a blood pressure calculation unit that calculates at least one of an absolute value and a trend of change of the systolic blood pressure (SBP) of the measurement subject by applying the second pulse wave transmission time (PTT) and unique parameter values (a, y0) of the measurement subject to a PTT-SBP relationship,
wherein unique parameter values (a, y0) of the measurement subject are acquired by analyzing data regarding pulse transit time (PTT) and a systolic blood pressure (SBP) of a patient group, extracting a correlation between the parameters (a, y0) regarding the PTT-SBP (Pulse Transit Time-Systolic Blood Pressure) relationship (SBP=a PTT−1+SBP), and applying the correlation between the parameters (a, y0) and data regarding a first pulse transit time (PTT) and the systolic blood pressure (SBP) of the measurement subject to the PTT-SBP relationship.

15. A computer-readable recording medium comprising:
a program for executing the method according to claim 1.

16. A computer-readable recording medium comprising:
a program for executing the method according to claim 2.

17. A computer-readable recording medium comprising:
a program for executing the method according to claim 3.

18. A computer-readable recording medium comprising:
a program for executing the method according to claim 4.

19. A computer-readable recording medium comprising:
a program for executing the method according to claim 5.
